(19) United States
(12) Patent Application Publication
(10) Pub. No.: US 2026/0083987 A1
(43) Pub. Date: Mar. 26, 2026

(54) METHOD AND APPARATUS TO FACILITATE OPTIMIZING A RADIATION TREATMENT PLAN REMOTELY

(71) Applicant: Varian Medical Systems, Inc., Palo Alto, CA (US)

(72) Inventors: Corinna Maier-Matic, München (DE); Magdalena Constantin, Los Altos, CA (US); Simona Sadlonova, Myjava (SK); Enrico Lyb, Tokyo (JP); Carolin Keilholz, Kirchensittenbach (DE); Stephen Thompson, Marina, CA (US); Tomasz Morgas, Basel (CH); Marco Lessard, Trois-Rivieres (CA); Dipesh Shah, Fremont, CA (US)

(21) Appl. No.: 19/339,716

(22) Filed: Sep. 25, 2025

Related U.S. Application Data
(60) Provisional application No. 63/699,341, filed on Sep. 26, 2024.

(51) Int. Cl. A61N 5/10

(52) U.S. Cl. CPC A61N 5/1071; A61N 2005/1074

(57) ABSTRACT

These various embodiments can serve to facilitate optimizing a radiation treatment plan to administer therapeutic radiation to a particular patient using a particular radiation treatment platform (that may include, for example, a medical linear accelerator) located at a facility having a radiation treatment platform user interface. The radiation treatment platform user interface is coupled to a remote user interface via a secure remote connection that does not permit the remote user interface to operate any radiation delivery capability of the treatment platform nor to execute movement of any of the dynamical components and to then conduct two-way communications (which may be encrypted if desired) between the radiation treatment platform user interface and the remote user interface regarding a radiation treatment plan to administer therapeutic radiation to the particular patient using the particular radiation treatment platform, wherein the two-way communications can include a final approval of an optimized radiation treatment plan.

### CROSS-REFERENCE TO RELATED APPLICATION

This application claims the benefit of U.S. Provisional Application No. 63/699,341, filed Sep. 26, 2024, which is hereby incorporated herein by reference in its entirety.

### TECHNICAL FIELD

These teachings relate generally to treating a patient's planning target volume with energy pursuant to an energy-based treatment plan and more particularly to optimizing and approving an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

For a variety of reasons, an optimized radiation treatment plan will typically require verification and/or approval shortly prior to administering the treatment. The latter helps to ensure that any changes to the patient's presentation are properly taken into account. Accordingly, a corresponding workflow may include computed tomography cone beam scans, development of a three-dimensional model of the day, and ensuing verification and sign-off steps. These steps typically require highly specialized and trained operators. And only a limited number of persons (perhaps only one) may have the authority to sign off on plan adaptations.

### BRIEF DESCRIPTION OF DRAWINGS

The above needs are at least partially met through provision of the method and apparatus to facilitate optimizing a radiation treatment plan described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a block diagram as configured in accordance with various embodiments of these teachings;

FIG. 4 comprises a block diagram as configured in accordance with various embodiments of these teachings;

FIG. 5 comprises a screenshot as configured in accordance with various embodiments of these teachings;

FIG. 6 comprises a screenshot detail view as configured in accordance with various embodiments of these teachings; and

FIG. 7 comprises a signal flow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments can serve to facilitate optimizing a radiation treatment plan to administer therapeutic radiation to a particular patient using a particular radiation treatment platform (that may include, for example, a medical linear accelerator) located at a facility having a radiation treatment platform user interface. In those regards, these teachings can provide for operably coupling the radiation treatment platform user interface to a remote user interface via a remote connection that does not permit the remote user interface to operate any radiation delivery capability of the particular radiation treatment platform (such as, for example, not permitting the remote user interface to operate a medical linear accelerator) and to then conduct two-way communications (which may be encrypted if desired) between the radiation treatment platform user interface and the remote user interface regarding a radiation treatment plan to administer therapeutic radiation to the particular patient using the particular radiation treatment platform, wherein the two-way communications can include a final approval of an optimized radiation treatment plan.

By one approach, the aforementioned remote connection may comprise a keyboard, video, mouse over Internet Protocol (KVM over IP) remote connection (which may comprise, if desired, a stand-alone hardware network element).

By one approach, the aforementioned remote connection can comprise a signaling protocol that requires the remote user interface to provide an authentication code to the radiation treatment platform user interface before allowing the conducting of the two-way communication between the radiation treatment platform user interface and the remote user interface. Provision of that authentication code to a user of the remote user interface may, for example, be initiated by the radiation treatment platform user interface. By one approach, that authentication code is provided to the user of the remote user interface via a communications path other than the remote connection. By one approach, the remote user interface may conduct the aforementioned two-way communications via a browser.

These teachings will accommodate a wide variety of content to be conveyed via the aforementioned two-way communications. Content examples include, but are not limited to, one or more of patient imagery, patient segmentation information, at least one treatment plan option, therapeutic dose distribution information, one or more dose volume histograms for various patient volumes, optimization objectives information (including changes to an optimization objective being communicated from the remote user interface to the radiation treatment platform user interface), and/or clinical goal(s) information.

The aforementioned final approval can comprise a necessary approval before therapeutic radiation can be administered to the particular patient using the optimized radiation treatment plan.

These teachings are highly flexible in practice and will accommodate a variety of modifications and/or supplemental features. As one example in these regards, these teachings will accommodate operably coupling the aforementioned remote user interface to a further remote user interface via an extended remote connection wherein the further remote user interface is provided with fewer communications capabilities than the remote user interface to communicate to the radiation treatment platform user interface. As one example in the latter regards, the further remote user interface may exclude any opportunity to source the aforementioned final approval of an optimized radiation treatment plan.

By one approach, these teachings can comprise a non-transitory computer-readable medium having a computer program that itself comprises instructions that, when the computer program is executed by a computer, causes the computer to carry out any one or more of the steps, actions, and/or functions described herein. By one approach, for example, the computer program, when executed by a computer, can facilitate operably coupling the radiation treatment platform user interface to a remote user interface via a remote connection and to then conduct two-way communications (which may be encrypted if desired) between the radiation treatment platform user interface and the remote user interface regarding a radiation treatment plan to administer therapeutic radiation to the particular patient using the particular radiation treatment platform, wherein the two-way communications can include a final approval of an optimized radiation treatment plan. By one approach the aforementioned remote connection can comprise a signaling protocol that requires the remote user interface to provide an authentication code to the radiation treatment platform user interface before allowing the conducting of the two-way communication between the radiation treatment platform user interface and the remote user interface. Provision of that authentication code to a user of the remote user interface may, for example, be initiated by the radiation treatment platform user interface. By one approach, the computer program will support the remote user interface conducting the aforementioned two-way communications via a browser.

Existing approaches that require input and/or approval by either a specified person or a person having a particular credential or authorization serve to help ensure the quality of the radiation treatment to be administered to a particular patient. For any number of reasons, however, the necessary or otherwise useful person may not be immediately physically available in the vicinity of the radiation treatment platform user interface. This oft-experienced situation then necessarily leads to delay. That delay can be uncomfortable for the patient and can reduce the productivity and throughput for the radiation treatment platform itself (which can lead to reduced availability to other patients who are awaiting their own treatment). The present teachings can significantly ameliorate the temporal downside of supporting such a workflow by making it easier for necessary personnel to play their part in the pre-treatment process (including, but not limited to, providing a requisite final approval of an optimized radiation treatment plan).

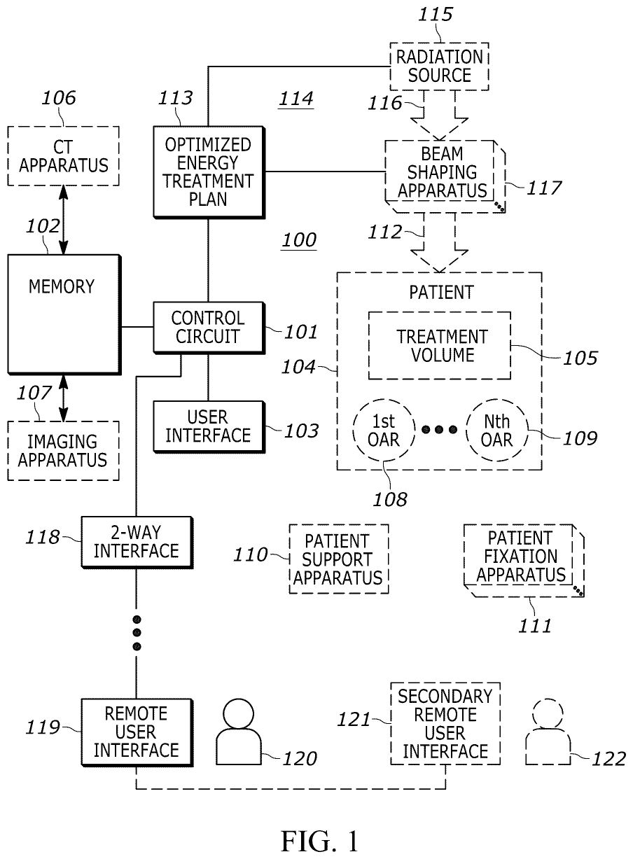

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as optimization information for a particular patient, imagery information for the particular patient, and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

In this illustrative example the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons. Other possibilities include circular accelerators or proton systems. (If desired, these teachings can also be applied in conjunction with the radiation sources that are associated with brachytherapy.)

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

In this illustrative example the control circuit 101 also communicatively couples to a two-way interface 118 that can serve as a remote connection to a remote user interface 119. As used herein, the word “remote” refers to a location that is at least in a different room than where the radiation treatment platform 114 and/or the aforementioned user interface 103 is located. This remote location may be elsewhere in a same building that contains the radiation treatment platform 114 and the user interface 103 or the remote location may be located in another building. The latter may be in a same clinic facility or campus, or not. There are generally no current practical limitations with respect to how distant the remote user interface 119 may be.

So configured, a remote user 120 can utilize the remote user interface 119 as described herein to participate in one or more steps of the pre-treatment workflow, including providing a necessary final approval to an optimized radiation treatment plan 113.

These teachings will also accommodate operably coupling the remote user interface 119 to one or more secondary remote user interfaces 121 as described herein. So configured, the aforementioned remote user 120 can, in turn, communicate with another remote user 122 to garner, for example, expert input from the latter. Using this approach, any number of additional remote users could be included, either serially or collectively.

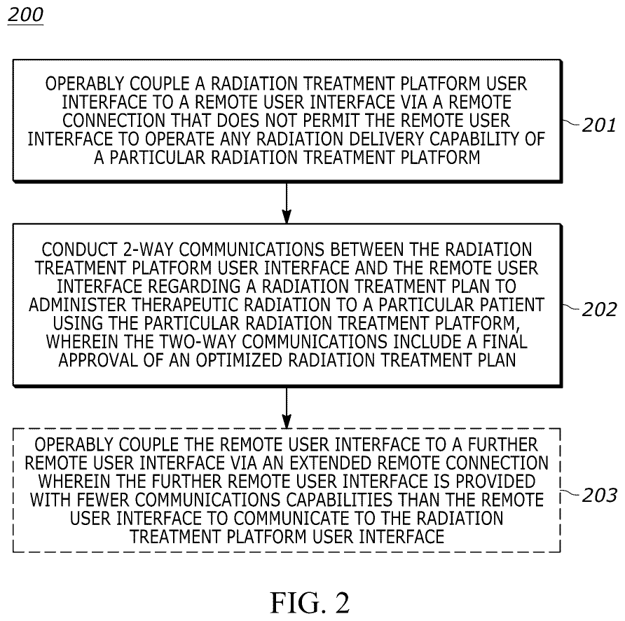

Referring now to FIG. 2, a process 200 that can be carried out, for example, in conjunction with the above-described application setting will be described. Generally speaking, this process 200 serves to facilitate optimizing a radiation treatment plan to administer therapeutic energy 112 (such as radiation) to a particular patient 104 using a particular radiation treatment platform 114 located at a facility having a radiation treatment platform user interface 103 (for example, as described above). It will be appreciated that these teachings can also serve to optimize imaging parameters and/or to otherwise conduct patient image acquisition, particularly during a day-of-treatment workflow.

At block 201, this process 200 provides for operably coupling the radiation treatment platform user interface 103 to a remote user interface 119 via a remote connection that does not permit the remote user interface 119 to operate any radiation delivery capability of the particular radiation treatment platform 114. By way of example, when the particular radiation treatment platform 114 includes a medical linear accelerator, the remote connection does not permit the remote user interface 119 to operate that medical linear accelerator. In particular, a remote user 120 is not able, inadvertently or intentionally, to switch the radiation source 115 into a beam-on operating state (and/or to execute movement on any of the movable axes of the treatment delivery platform (such as a patient support surface, a collimator, a gantry, and/or beam limiting/shaping devices).

By one approach, the remote connection can comprise a keyboard, video, mouse over Internet Protocol (KVM over IP) remote connection. KVM over IP comprises a known technology and can comprise a software adjunct to a computer user interface or can comprise a stand-alone hardware network element. KVM over IP typically serves to allow a user to remotely control one or more computers from a remote set of interface devices-such as a keyboard, video monitor, and mouse-over a network using the Internet Protocol. This means that an individual can access and manage servers or workstations located in different physical locations as if they were directly in front of them, without the need for actual physical presence. The technology typically serves to facilitate the transmission and reception of video signals and the transmission of input device signals (for example, from a keyboard, mouse, trackball, touchpad, touchscreen and so forth) and then transmit that content over a network, allowing for real-time interaction with the remote systems.

By one approach, some or all of the foregoing two-way communications can be encrypted (using, for example, public key encryption).

So configured, the remote user interface 119 can receive and present to the remote user 120 a variety of useful content. Examples include, but are not limited to:
patient imagery (such as, but not limited to, computed tomography images and any other patient reference images such as kilovoltage and/or megavoltage cone beam computed tomography images (including images that were captured previously as well as images that were captured pre-treatment on the day-of-treatment));
patient anatomy segmentation information (typically comprising medical imaging scans have the various organs and tissues within the patient's body delineated or otherwise outlined);
at least one treatment plan option (such as, for example, an intensity modulated radiation therapy plan, a volumetric modulated arc therapy plan, or a stereotactic radiosurgery plan).
dose distribution information (such as a statement or table that correlates different patient volumes with different minimum or maximum radiation doses in Gy's);
a dose volume histogram (which typically represent three-dimensional dose distributions in a graphical two-dimensional format (the three-dimensional dose distributions being created, for example, in a computerized radiation-treatment planning system based on a three-dimensional reconstruction of an X-ray computed tomography scan and study); the “volume” referred to in DVH analysis can be, for example, the radiation-treatment target, a healthy organ located near such a target, an arbitrary structure, and so forth);
optimization objectives information including changes made to an existing optimization objective by a remote user 120 via the remote user interface 119 (with at least some optimization objectives typically being derived from a prescribing physician's treatment instructions that specify clinical metrics for goals pertaining at least to the patient's treatment volume and sometimes to adjacent so-called organs-at-risk (OAR) as well; generally speaking, optimization objectives are often used by the optimizer as the building blocks of a cost function); and/or
clinical goals information (such as the treatment goals prescribed by, for example, an attending oncologist; examples of clinical goals include, but are not limited to, goals regarding the dose distributions to be achieved with respect to a target volume, one or more organs-at-risk (OAR) in the vicinity of the target volume, or other specified or unspecified normal tissues; by their very nature, clinical goals are typically agnostic with respect to what physical radiation treatment platform serves to administer the radiation).

Although the described arrangement will not allow a remote user 120 to operate any radiation delivery capability of the radiation treatment platform 114 (including, in particular, switching the radiation source 115 into an “on” state and/or movement of any of the dynamical axes of the treatment delivery platform), this arrangement will specifically allow transmission from the remote location to the facility location of a final necessary approval of an optimized radiation treatment plan 113 from a person who is authorized to issue such an approval. The latter can occur, for example, upon the remote user 120 having logged in or otherwise established themselves as an appropriately authorized person to issue such an approval.

At block 202, this process 200 then provides for conducting two-way communications between the radiation treatment platform user interface 103 and the remote user interface 119 regarding a radiation treatment plan to administer therapeutic radiation 112 to the particular patient 104 using the particular radiation treatment platform 114, wherein the two-way communications can include a final approval of an optimized radiation treatment plan 113.

These two-way communications can be conducted, back and forth, in at least near real time. This can be especially so when the communications link is rated to support rapid sharing of voluminous medical imagery content.

The aforementioned final approval can have the form of a legally binding approval if desired. In particular, these teachings will support having a remote user 120 who is an authorized medical expert sign-off on an adapted treatment plan in a legally binding manner. By one approach, that remote expert can be authenticated by the system and the approval can be logged and documented in such a manner that it can be transmitted to an electronic medical record system through an open or proprietary interface for retention of the record and for evidence to support needs such as reimbursement. This sign-off capability can comprise a part of the remote control capability of the local client but a feature and responsibility of adaptive software. If desired, a different communication channel can be used for this purpose in lieu of or in addition to a KVM over IP channel.

By one approach, these two-way communications can include facilitating the remote optimization of computed tomography scan parameters as part of a daily image guided radiation therapy session or to otherwise conduct patient image acquisition. These teachings can also serve the use case of facilitating a remote video interface to ensure proper patient setup/positioning for SRS treatment prior to authorize a beam-on state. Similarly, these teachings are capable of transmitting a video camera feed for a camera installed in the radiation treatment room that shows the patient setup and positioning during stereotactic radiosurgery.

As shown at optional block 203 these teachings will also accommodate operably coupling the remote user interface 119 to a further remote user interface (such as the secondary remote user interface 121 described above) via an extended remote connection wherein the further remote user interface is provided with fewer communications capabilities than the remote user interface 119 as regards communicating with the radiation treatment platform user interface. In particular, in addition to lacking a capability of switching the radiation source 116 to an “on” state, the secondary remote user interface 121 can also be denied the opportunity to source the above-described final approval of an optimized radiation treatment plan 113.

Although lacking such capabilities, such an extended opportunity to permit the remote user 120 to communicate with one or more other remote users 122 can allow the former to avail themselves of expertise that they may themselves lack. As one illustrative example, the optimized radiation treatment plan 113 may encompass a particular organ-at-risk regarding which the remote user 120 may be less familiar. Using the secondary remote user interface 121 opportunity, the remote user 120 could effectively conference in a subject matter expert for the purpose of gaining that person's perspective and expert opinion on this one particular aspect of the plan.

Further details that comport with these teachings will now be presented. It will be understood that the specific details of these examples are intended to serve an illustrative purpose and are not intended to suggest any particular limitations with respect to these teachings.

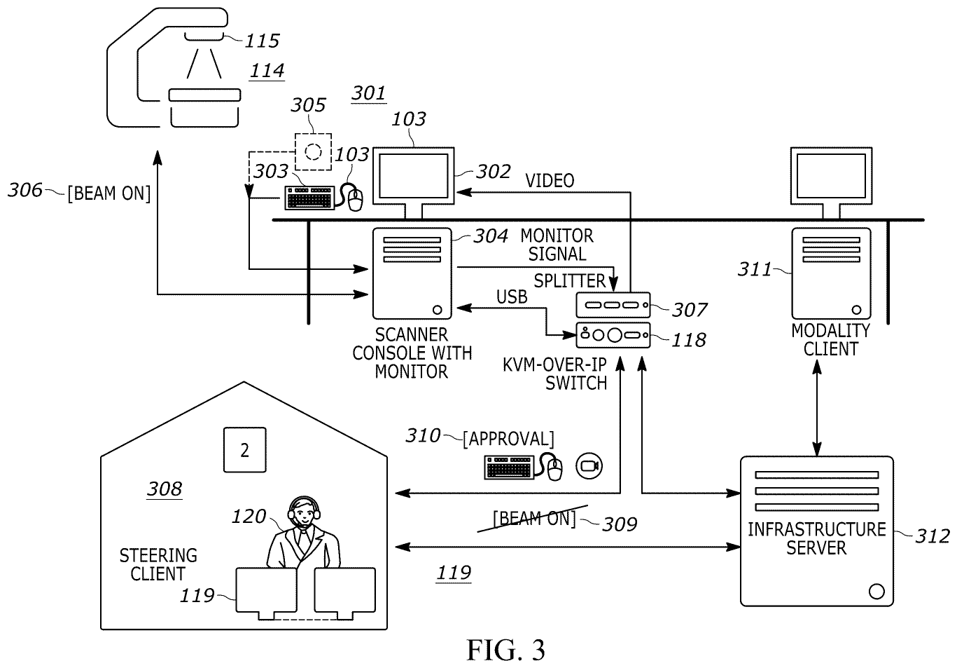

FIG. 3 presents an illustrative example of a sample instantiation of these teachings. In this example, a radiation treatment platform 114 having a radiation source 115 is located at a first location 301 such as a treatment clinic. This first location 301 also includes the aforementioned user interface 103 that includes at least one monitor 302 and keyboard/mouse 303 that are communicatively coupled to a scanner console (or other appropriate treatment device console) 304. The aforementioned radiation source 115 may be switched to a beam-on state via a beam-on signal 306 that is sourced from the aforementioned user interface 103 components and/or, if desired, via a dedicated hardware button 305. The foregoing components are known in the art. As the present teachings are not overly sensitive to any particular choices in these regards, further elaboration regarding these components is not provided here for the sake of brevity.

The two-way interface 118 in this example comprises a KVM over IP switch. This module communicably couples via a universal serial bus (USB) connection to the scanner console 304 and also operably couples to a monitor signal splitter that splits video content sourced by the scanner console 304 to both the aforementioned monitor 302 and to the KVM over IP switch.

At a second location 308, which second location 308 is remote from the first location 301, a remote user 120 utilizes a remote user interface 119 to communicate video content as well as keyboard/cursor control signals with the scanner console 304. So configured, the remote user 120 can view the same information regarding the patient and the radiation treatment plan as would be the case if they were physically present at the first location 301. And, while the remote user 120 is precluded from sourcing a direct beam-on command to the radiation source 115 (as denoted by reference numeral 309), the remote user 120 is able to convey a final approval (as denoted by reference numeral 310) that is necessary to permit the radiation treatment to proceed for the patient.

As illustrated, this particular example also makes use of a modality client 311 that can serve to grant interactive access to the radiation treatment platform 114 to the remote user 120 and to provide a communication (audio/video/chat) channel to a steering client at the remote user interface 119.

Also illustrated is an SVC server (Storage Area Network (SAN) Volume Controller server) 312. (It will be appreciated that such a server could be deployed essentially anywhere, including in the cloud.) This server 312 can provide system administration and management services, and can facilitate enabling and managing encrypted/secured communication between the local and remote clients. This server 312 may, if desired, obtain user credentials and authentication from external sources (not shown) following recognized open-source or proprietary standards. Or, in the alternative, may host its own secure user administration and management.

So configured, these teachings can support the following illustrative workflow. The details of this example are intended to provide an illustrative example and are not intended to suggest any limitations with respect to these teachings.

Reference data can be loaded by the treatment platform console software when a patient is present with the intent of administering radiation treatment. The reference data may be, for example, information regarding the session and/or patient that is relevant for the treatment plan. For instance, the reference data may include a fast computed tomography scan done at the local site prior to the treatment. Using such reference data may be optional, depending upon circumstances and the particulars of the application setting and the patient's presentation. The console software can be configured to display a representation for treatment planning of the session patient for display at the local site with a monitor or the like.

The aforementioned content, such as the representation, can be transmitted to a remote site using a KVM over IP switch. This example assumes that the network bandwidth for the connection between the local and remote clients and also the display monitors at the remote client are meeting a set of pre-defined specifications for medical imaging and radiotherapy applications. This example also assumes that appropriate security measures are being observed.

At the remote site, the transmitted content (such as the aforementioned representation) is shown on the remote display monitors to a remote medical expert.

The remote medical expert may perform user inputs directed to reviewing and adapting the treatment imaging session and/or the treatment plan. Such user inputs can be transmitted to the local client via the KVM over IP switch where those inputs can be used to interactively modify, review, and ultimately approve the optimized parameters of the treatment session.

Accordingly, such a workflow can support obtaining an adapted treatment plan based on expert input from a remote location, which adapted treatment plan can then be administered at the local site. In addition, an adapted treatment may be technically approved from the remote client upon completing a quality assurance session and/or an adapted treatment may be clinically approved from a remote client upon reviewing the clinical goals of the plan.

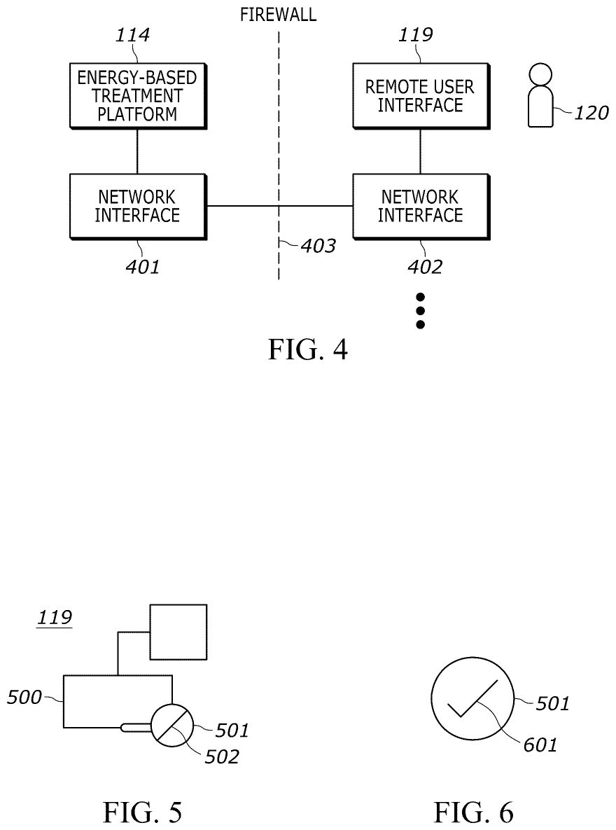

FIG. 4 presents an alternative approach to using the KVM-over-IP switch 118 (and it's associated supporting components and circuitry). By this approach, the energy-based treatment platform 114 (such as the aforementioned console 304) operably couples to a network interface 401 that in turn communicates with a remote user interface 119 via it's own network interface 402. These teachings will accommodate any of a variety of network interfaces, it only being generally required that the two network interfaces 401 and 402 be communicatively compatible with one another (and/or any intervening network(s)). If desired, one or more security firewalls 403 may be utilized to further guard the security of the energy-based treatment platform 114.

By one approach, the aforementioned scanner monitor 302 can run and present a user program that is configured to employ the energy-based treatment platform 114 to administer therapeutic energy to a patient. By one approach, the latter can include presenting on the monitor 302 a graphic element 500 that can serve to enable and disable two-way communications between the energy-based treatment platform 114 and the remote user interface 119.

In this illustrative example, this graphic element 500 includes a circle 501 having a diagonal line 502 disposed therewith. So configured, the graphic element 500 signals that such two-way communications are not presently enabled. By asserting this graphic element 500 (using, for example, a mouse click or a finger touch), a user can enable remote access capability and the aforementioned circle 501 can now encompass and present a check mark 601 to indicate such enablement. (The foregoing examples presented in FIGS. 5 and 6 are only intended to serve an illustrative purpose. These teachings will accommodate any of a variety of graphic and/or textual elements to serve in the foregoing regards. These teachings will also accommodate, if desired, placing the ability to enable remote access capability within a sub-menu or the like.)

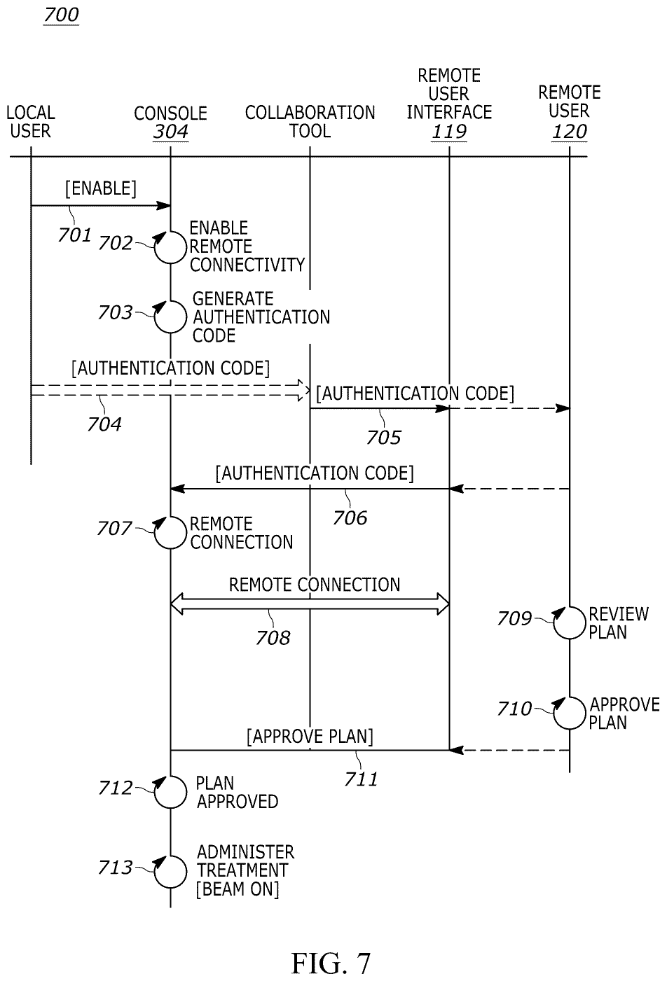

FIG. 7 presents an illustrative example of a signaling protocol 700 that can be utilized in conjunction with the architectural example presented in FIG. 4. This protocol 700 begins when a user local to the energy-based treatment platform 114 enables (at reference numeral 701) a capability of the console 304 to engage in two-way communications with a remote user (for example, by selectively asserting the aforementioned graphic element 500 described above). In response, the console 304 enables (at reference numeral 702) the selected remote connectivity capability and generates (at reference numeral 703) a corresponding authentication code.

These teachings will accommodate any of a variety of ways of generating that authentication code. Generally speaking, it may be preferable for that authentication code to be relatively unique and to be randomly generated (or otherwise selected) at the time of need.

At reference numeral 704, that authentication code is communicated (either by being sourced by the local user or by the console 304) via a collaboration tool (such as an e-mail system, an instant messaging service, a facility teamwork service such as TEAMS, or the like) that passes the authentication code (at 705) to the remote user interface 119 and/or to the remote user 120 as appropriate.

For the sake of an illustrative example, it is presumed here that the remote user interface 119 is using a browser of choice to communicate with the console 304 (for example, via the above-described network interfaces 401 and 402). A browser is a software application that enables a user to access, retrieve, and display content from the World Wide Web or other networked resources. Such a program interprets and renders data transmitted using standard protocols (such as HTTP or HTTPS) and formats (such as HTML, CSS, and JavaScript), presenting the information in a human-readable form. A browser also provides an interface for user interaction, including navigation, input submission, and execution of client-side scripts, thereby facilitating communication between the user and remote programs or services.

At reference numeral 706, the remote user 120/remote user interface 119 forwards that authentication code back to the console 304. In response to receiving the authentication code, the console 304 can confirm the veracity of the received code and, in response, initiate the remote connection at reference numeral 707. The latter, in turn, results in opening a two-way remote connection 708 with the remote user interface 119.

So configured, the remote user 120 can interface and interact with the console 304 as described above. The remote user 120 can, for example, review the energy-based treatment plan as indicated at reference numeral 709.

As before, however, this signaling protocol 700 will not support the remote user 120 activating the radiation source 116 of the energy-based treatment platform 114. Instead, activation of the radiation source can only be done by someone physically present at the radiation treatment platform 114.

This protocol 700 will, however, permit the remote user 120 to officially approve the energy-based treatment plan (as denoted by reference numeral 710) and to send a message or signal 711 to the console 304 indicating such approval. In response, the console 304 can flag or otherwise update the status of the energy-based treatment plan as being officially and finally approved (as denoted by reference numerals 712), following which treatment can be administered (as denoted at reference numeral 713) and the radiation source can be switched to a “beam on” state as part of administering that treatment.

So configured, these teachings can provide remote assistance during radiation treatment planning and adaptation steps. In particular, these teachings provide for enabling the control of treatment plan adaptation software running at a (local) treatment facility from a remote location. Although these teachings provide for highly effective and broad-based access to information, these teachings are not tied to software releases specific to particular radiation-delivery modalities and therefore will not typically be subject to release restrictions or cycles.

Those skilled in the art will recognize that a wide variety of modifications, alterations, and combinations can be made with respect to the above-described embodiments without departing from the scope of the invention.

As one example in these regards, these teachings will accommodate being combined with an artificial intelligence capability. For example, additional assistance may be provided by an artificial intelligence tool at the remote operator site that captures displayed screen information from the user interface 103 and analyzes that displayed information for relevant actions such as matching a verification computed tomography image to the initial session computed tomography image, and/or improvement of the treatment plan (as regards, for example, segmentation and/or planning options) that can be suggested to the remote user.

As another example in these regards, these teachings can be employed in the context of emergency treatments where a reference computed tomography image is not available for the patient. In such a case, the treatment delivery platform may be capable of performing cone beam computed tomography imaging for planning (CBCTp) purposes. In this situation, interactive access from a remote medical expert may be used to edit and optimize the image mode parameters for the CBCTp image session (also sometimes referred to as the patient simulation session). Moreover, interactive access from the remote medical expert may be used to identify and mark the treatment isocenter.

Accordingly, modifications, alterations, and combinations in these regards are to be viewed as being within the ambit of the inventive concept.

Further aspects of the invention are provided by the subject matter of the following clauses (where it will be understood that any of these clauses can be combined with any one of more of the other clauses as appropriate).

Clause 1. A method to facilitate optimizing a radiation treatment plan to administer therapeutic radiation to a particular patient using a particular radiation treatment platform located at a facility having a radiation treatment platform user interface, the method comprising: operably coupling the radiation treatment platform user interface to a remote user interface via a remote connection that does not permit the remote user interface to operate any radiation delivery capability of the particular radiation treatment platform; conducting two-way communications between the radiation treatment platform user interface and the remote user interface regarding a radiation treatment plan to administer therapeutic radiation to the particular patient using the particular radiation treatment platform, wherein the two-way communications include a final approval of an optimized radiation treatment plan.

Clause 2. The method of clause 1 wherein the particular radiation treatment platform includes a medical linear accelerator or other ionizing radiation source.

Clause 3. The method of clause 2 wherein not permitting the remote user interface to operate any radiation delivery capability of the particular radiation treatment platform comprises not permitting the remote user interface to operate the medical linear accelerator.

Clause 4. The method of any of clauses 1 through 3 wherein the remote connection comprises a keyboard, video, mouse over Internet Protocol (KVM over IP) remote connection.

Clause 5. The method of clause 4 wherein the KVM over IP remote connection comprises a stand-alone hardware network element.

Clause 6. The method of any of clauses 1 through 5 wherein conducting the two-way communications comprises conducting at least some of the two-way communications in an encrypted manner.

Clause 7. The method of any of clauses 1 through 6 wherein the two-way communications include at least one of: patient imagery; patient anatomy segmentation information; at least one treatment plan option; dose distribution information; a dose volume histogram; optimization objectives information; clinical goals information.

Clause 8. The method of any of clauses 1 through 7 wherein the two-way communications include changes to an optimization objective being communicated from the remote user interface to the radiation treatment platform user interface.

Clause 9. The method of any of clauses 1 through 8 wherein the final approval comprises a necessary approval before therapeutic radiation can be administered to the particular patient using the optimized radiation treatment plan.

Clause 10. The method of any of clauses 1 through 9 further comprising: operably coupling the remote user interface to a further remote user interface via an extended remote connection wherein the further remote user interface is provided with fewer communications capabilities than the remote user interface to communicate to the radiation treatment platform user interface.

Clause 11. An apparatus to facilitate optimizing a radiation treatment plan to administer therapeutic radiation to a particular patient using a particular radiation treatment platform located at a facility having a radiation treatment platform user interface, the apparatus comprising: a remote connection configured to operably couple the radiation treatment platform user interface to a remote user interface, wherein the remote connection does not permit the remote user interface to operate any radiation delivery capability of the particular radiation treatment platform, and wherein the remote connection is configured to conduct two-way communications between the radiation treatment platform user interface and the remote user interface regarding a radiation treatment plan to administer therapeutic radiation to the particular patient using the particular radiation treatment platform, wherein the two-way communications include a final approval of an optimized radiation treatment plan.

Clause 12. The apparatus of clause 11 wherein the particular radiation treatment platform includes a medical linear accelerator.

Clause 13. The apparatus of clause 12 wherein not permitting the remote user interface to operate any radiation delivery capability of the particular radiation treatment platform comprises not permitting the remote user interface to operate the medical linear accelerator.

Clause 14. The apparatus of any of clauses 11 through 13 wherein the remote connection comprises a keyboard, video, mouse over Internet Protocol (KVM over IP) remote connection.

Clause 15. The apparatus of clause 14 wherein the KVM over IP remote connection comprises a stand-alone hardware network element.

Clause 16. The apparatus of any of clauses 11 through 15 wherein the two-way communications comprise, at least in part, encrypted communications.

Clause 17. The apparatus of any of clauses 11 through 16 wherein the two-way communications include at least one of: patient imagery; a dose volume histogram; optimization objectives information; clinical goals information; patient segmentation information; at least one treatment plan option; dose distribution information.

Clause 18. The apparatus of any of clauses 11 through 17 wherein the two-way communications include changes to an optimization objective being communicated from the remote user interface to the radiation treatment platform user interface.

Clause 19. The apparatus of any of clauses 11 through 18 wherein the final approval comprises a necessary approval before therapeutic radiation can be administered to the particular patient using the optimized radiation treatment plan.

Clause 20. The apparatus of any of clauses 11 through 19 wherein the remote user interface is further configured to operably couple to a further remote user interface via an extended remote connection wherein the further remote user interface is provided with fewer communications capabilities than the remote user interface to communicate to the radiation treatment platform user interface.

Clause 21. A non-transitory computer-readable medium having a computer program that itself comprises instructions that, when the computer program is executed by a computer, causes the computer to carry out any one or more of the steps, actions, and/or functions described herein.

Clause 22. The non-transitory computer-readable medium of clause 21 wherein the computer program, when executed by the computer, facilitates operably coupling a radiation treatment platform user interface to a remote user interface via a remote connection.

Clause 23. The non-transitory computer-readable medium of clause 22 wherein the computer program, when executed by the computer, facilitates conducting two-way communications between the radiation treatment platform user interface and the remote user interface regarding a radiation treatment plan to administer therapeutic radiation to a particular patient using a particular radiation treatment platform.

Clause 24. The non-transitory computer-readable medium of clause 23 wherein the two-way communications can include a final approval of an optimized radiation treatment plan.

Clause 25. The non-transitory computer-readable medium of any of clauses 21 through 24 the remote connection comprises a signaling protocol that requires the remote user interface to provide an authentication code to the radiation treatment platform user interface before the conducting of the two-way communication between the radiation treatment platform user interface and the remote user interface is allowed.

Clause 26. The non-transitory computer-readable medium of clause 25 wherein the computer program, when executed by the computer, facilitates provision of the authentication code to a user of the remote user interface.

Clause 27. The non-transitory computer-readable medium of any of clauses 21 through 26 wherein the computer program, when executed by the computer, supports the remote user interface conducting the aforementioned two-way communications via a browser.

1. A method to facilitate optimizing a radiation treatment plan to administer therapeutic radiation to a particular patient using a particular radiation treatment platform located at a facility having a radiation treatment platform user interface, the method comprising:
operably coupling the radiation treatment platform user interface to a remote user interface via a remote connection that does not permit the remote user interface to operate any radiation delivery capability of the particular radiation treatment platform;
conducting two-way communications between the radiation treatment platform user interface and the remote user interface regarding a radiation treatment plan to administer therapeutic radiation to the particular patient using the particular radiation treatment platform, wherein the two-way communications include a final approval of an optimized radiation treatment plan.

2. The method of claim 1 wherein the particular radiation treatment platform includes a medical linear accelerator.

3. The method of claim 2 wherein not permitting the remote user interface to operate any radiation delivery capability of the particular radiation treatment platform comprises not permitting the remote user interface to operate the medical linear accelerator.

4. The method of claim 1 wherein the remote connection comprises a keyboard, video, mouse over Internet Protocol (KVM over IP) remote connection.

5. The method of claim 4 wherein the KVM over IP remote connection comprises a stand-alone hardware network element.

6. The method of claim 1 wherein the remote connection comprises a signaling protocol that requires the remote user interface to provide an authentication code to the radiation treatment platform user interface before allowing the conducting of the two-way communications between the radiation treatment platform user interface and the remote user interface.

7. The method of claim 6 wherein provision of the authentication code to a user of the remote user interface is initiated by the radiation treatment platform user interface.

8. The method of claim 7 wherein the authentication code is provided to the user of the remote user interface via a communications path other than the remote connection.

9. The method of claim 1 wherein the remote user interface conducts the two-way communications via a browser.

10. The method of claim 1 wherein conducting the two-way communications comprises conducting at least some of the two-way communications in an encrypted manner.

11. The method of claim 1 wherein the two-way communications include at least one of:
patient imagery;
patient segmentation information;
at least one treatment plan option;
dose distribution information;
a dose volume histogram;
optimization objectives information;
clinical goals information.

12. The method of claim 1 wherein the two-way communications include changes to an optimization objective being communicated from the remote user interface to the radiation treatment platform user interface.

13. The method of claim 1 wherein the final approval comprises a necessary approval before therapeutic radiation can be administered to the particular patient using the optimized radiation treatment plan.

14. The method of claim 1 further comprising:
operably coupling the remote user interface to a further remote user interface via an extended remote connection wherein the further remote user interface is provided with fewer communications capabilities than the remote user interface to communicate to the radiation treatment platform user interface.

15. An apparatus to facilitate optimizing a radiation treatment plan to administer therapeutic radiation to a particular patient using a particular radiation treatment platform located at a facility having a radiation treatment platform user interface, the apparatus comprising:
a remote connection configured to operably couple the radiation treatment platform user interface to a remote user interface, wherein the remote connection does not permit the remote user interface to operate any radiation delivery capability of the particular radiation treatment platform, and wherein the remote connection is configured to conduct two-way communications between the radiation treatment platform user interface and the remote user interface regarding a radiation treatment plan to administer therapeutic radiation to the particular patient using the particular radiation treatment platform, wherein the two-way communications include a final approval of an optimized radiation treatment plan.

16. The apparatus of claim 11 wherein the two-way communications include changes to an optimization objective being communicated from the remote user interface to the radiation treatment platform user interface.

17. The apparatus of claim 11 wherein the remote connection comprises a signaling protocol that requires the remote user interface to provide an authentication code to the radiation treatment platform user interface before allowing the conducting of the two-way communications between the radiation treatment platform user interface and the remote user interface.

18. The apparatus of claim 17 wherein provision of the authentication code to a user of the remote user interface is initiated by the radiation treatment platform user interface.

19. The apparatus of claim 17 wherein the authentication code is provided to the user of the remote user interface via a communications path other than the remote connection.

20. The apparatus of claim 11 wherein the remote user interface conducts the two-way communications via a browser.
